(19) United States
(12) Patent Application Publication
(10) Pub. No.: US 2008/0020068 A1
(43) Pub. Date: Jan. 24, 2008

(54) Use Of Trisubstituted Benzopyranones

(75) Inventors: Stefan Germer, Karlsruhe (DE); Hermann Hauer, Karlsruhe (DE); Egon Koch, Karlsruhe (DE); Karl Schotz, Durmersheim (DE)

Correspondence Address: EDWARDS ANGELL PALMER & DODGE LLP, P.O. BOX 55874, BOSTON, MA, 02205, US

(73) Assignee: Dr.Willmar Schwabe GmbH & Co. KG, Karlsruhe (DE)

(21) Appl. No.: 11/631,393

(22) PCT Filed: Jun. 30, 2005
(86) PCT No.: PCT/EP05/07051

§ 371 Date: Dec. 28, 2006

(30) Foreign Application Priority Data
Jul. 5, 2004 (DE) 102004032440.9

(51) Int. Cl. C07D 311/08; A61K 31/352; A61K 36/185

(52) U.S. Cl. 424/725; 514/457; 549/399

(57) ABSTRACT

The present invention relates to the use of trisubstituted benzopyranones for the treatment or propylaxis of pathological conditions associated with oxidative stress and/or inflammatory reactions as well as to novel trisubstituted benzopyranones and the physiologically acceptable salts thereof. The present invention further relates to plant extracts, medicaments, dietetic food products and pharmaceutical preparations containing these compounds.

The present invention relates to the use of trisubstituted benzopyranones for the treatment or prophylaxis of pathological conditions associated with oxidative stress and/or inflammatory reactions and to novel trisubstituted benzopyranones and the physiologically acceptable salts thereof. The present invention further relates to plant extracts, medicaments, dietetic food products and pharmaceutical preparations.

Free radicals are atoms or molecules having an unpaired electron in their outer orbit. For biological processes the most important free radical is molecular oxygen being capable of forming different metabolites by reduction. These metabolites are generally summarised under the collective term “reactive oxygen species” (ROS). Examples of ROS include the superoxide anion, hydroxyl radical, hydrogen peroxide, peroxide anion, singlet oxygen, hypochloride, nitrogen oxide and peroxy nitrite.

ROS are spontaneously formed by various biological processes. The so-called “respiratory burst” of leukocytes wherein after stimulating the cells with microorganisms, xenobiotics or endogenic substances the superoxide radical and other ROS are formed as reaction products starting from molecular oxygen by activating a membraneous NADPH oxidase is of particular importance. The respiratory burst is one of the most important mechanisms of the early unspecific immune defence and mainly serves for killing intruded infective agents and tumor cells. Additionally, ROS are mainly generated by a leakage of electrons resulting from unsufficiently coupled reactions. This occurs for example in the synthesis of prostaglandines and leukotrienes from arachidonic acid, during the mitochondrial respiration, by xanthinoxidase-catalysed oxidation of hypoxanthine under ischemic conditions or in the course of the cytochrome P450-mediated metabolisation of xenobiotics.

Whereas the respiratory burst is basically a desired reaction in the defence against infections, the increased and continuous formation of ROS is normally detrimental because the oxidative attack is not limited to intruding microorganisms, but also the body's own tissue is exposed to their toxic potential. This particularly applies to the non-infectious diseases, such as the increased formation of ROS in the course of autoimmune diseases, for degenerative diseases, during an ischemia or for the metabolisation of pharmaceutical agents. The undesired effects of free radicals and ROS are based on their interaction with nucleic acids (e. g. induction of DNA strand breaks), proteins (e. g. denaturation, inactivation of enzyme systems), carbohydrates (e. g. depolymerisation of hyaluronic acids) and particularly lipids (e. g. lipid peroxydation, lesion of membranes, formation of proinflammatory prostaglandins and leukotrienes).

After it has been postulated about 50 years ago that reactive oxygen species (ROS) are involved in the pathogenesis of various diseases, today it is considered certain that these molecules play an important role in the pathogenesis of numerous diseases, such as diabetes mellitus type I and II, inflammatory diseases (e. g. rheumatoid arthritis, asthma, colitis ulcerosa, psoriasis), bacterial and viral infections (e. g. influenza, AIDS, viral hepatitis), artherosclerosis, ischemias, neurologic diseases (e. g. Morbus Alzheimer, Morbus Parkinson and other neurodegenerative diseases), cataract, sickle cell anemia and tumor diseases, and that they are also further co-responsible for aging processes (A. Bendich (1994) in: B. Frei (ed.) “Natural Antioxidants in Human Health and Disease”, Academic Press, San Diego, p. 447; E. Peterhans (1997) J. Nutr. 127, 962 S; D. V. Parke (1999) in: T. K. Basu et al. (ed.) “Antioxidants in Human Health”, CAB International, p. 1).

The organism has various defence systems for the protection against the harmful effects of free radicals and ROS. These include vitamins (e. g. vitamin E and C) and other low-molecular compounds (e. g. glutathiones, uric acid), antioxidative enzymes (e. g. superoxide dismutase, catalase and glutathione peroxidase) as well as metal-binding proteins (e. g. transferrin, ceruloplasmin). However, the body's own antioxidative systems are frequently active during the initial phase of a pathological process only because the increased concentration of ROS formed in the progressing pathological process exceeds the capacity of the endogenic protection mechanisms by far.

Therefore, oxidative stress is considered to be a disproportion between the concentration of ROS and the antioxidative defence systems. Thus, due to the outstanding importance of ROS with respect to numerous diseases there is an extraordinary interest in substances having antioxidative properties that can be used in the prophylaxis and therapy of such pathological conditions.

Since ROS are of particular importance for inflammatory reactions and oxidative stress is frequently accompanied by an increased synthesis of proinflammatory eicosanoids (e. g. prostaglandines,-leukotrienes) and cytokines (e. g. IL-1, TNF-α, IL-6), there is particularly a demand for substances that exhibit antioxidative properties and additionally also prevent the formation of these inflammation mediators.

It is the object underlying the present invention to provide compounds for the treatment or prophylaxis of pathological conditions associated with oxidative stress and/or inflammatory reactions.

This object is solved by the use of compounds of general formula 1, 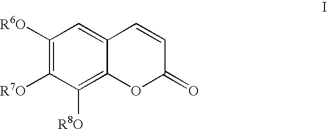
wherein the residues R⁶, R⁷ and R⁸ independently represent H or SO₃H, and the physiologically acceptable salts thereof for the treatment or prophylaxis of pathological diseases associated with oxidative stress and/or inflammatory reactions.

It has surprisingly been found that 6,7,8-Trihydroxy-2H-1-benzopyran-2-one (compound II) exhibits particularly advantageous pharmacological properties. In addition to potent antioxidative actions this compound also inhibits the synthesis of leukotrienes and prostaglandins as well as the synthesis of the proinflammatory cytokines IL-1β, TNF-α and IL-6. Thus, compound II is basically suitable for the treatment or prophylaxis of diseases accompanied by oxidative stress, such as diabetes mellitus type I and/or II, atherosclerosis and endothelial dysfunction, ischemias, neurological diseases (e. g. Morbus Alzheimer, Morbus Parkinson and other neurodegenerative diseases), cataract and tumor diseases. However, compound II is particularly advantageous for pathological diseases having an inflammatory component, such as rheumatoid arthritis, asthma, colitis ulcerosa, Morbus Crohn, psoriasis, neurodermitis and infections by bacteria, viruses (e. g. influenza, AIDS, viral hepatitis) and other pathogens (e. g. parasites, fungi and prions). Compound II has already been described in the literature (O. Kayser and H. Kolodziej, Phytochemistry 39, 1181-1185 (1995); S. Kumar, A. B. Ray, C. Konno, Y. Oshima and H. Hikino, Phytochemistry 27, 636-638 (1988); K. P. Lafte, O. Kayser, N. Tan, M. Kaloga and H. Kolodziej, Z. Naturforsch. 55c, 528-533 (2000)), however, phamacological effects of compound II are hitherto unknown. Compound II is contained in Pelargonium sidoides in a concentration of only 0.0004% (Kayser et al.; Latte et al., cf. above) and in Pelargonium reniforme in a concentration of only 0.02% (Latte et al., cf. above). It is to be concluded therefrom that compound II does not provide a considerable contribution to the biological efficacy of Pelargonium sidoides and reniforme in these low concentrations, respectively.

Therefore, the subject of the present invention is the use of compound II for the treatment or the prophylaxis of pathological conditions associated with oxidative stress and/or inflammatory reactions.

It is also possible to administer compound II in the form of sulfuric acid esters of general formula I because compound II is released from those compounds upon oral administration. For this reason also the compounds of general formula I are also suitable for the treatment or prophylaxis of the above-mentioned pathological conditions. Preferred compounds of general formula I are 6,7-dihydroxy-8-sulfooxy-2H-1-benzopyran-2-one (R⁶═R⁷═H; R⁸═SO₃H) and 7,8-dihydroxy-6-sulfooxy-1-benzopyran-2-one (R⁷═R⁸═H; R⁶═SO₃H). 6,8-Bis(sulfooxy)-7-hydroxy-2H-benzopyran-2-one (R⁶═R⁸═SO₃H; R⁷═H; compound III) is particularly preferred. The compounds of general formula I wherein at least one of the residues R⁶, R⁷ or R⁸ is an SO₃H residue are novel. Therefore, these compounds, and particularly compound III, as well as their use for the treatment or prophylaxis of pathological conditions associated with oxidative stress and/or inflammatory reactions are also a part of the present invention.

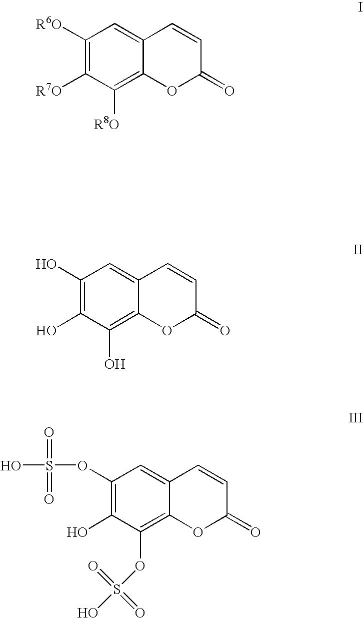

In general formula I the residues R⁶, R⁷ and R⁸ are independently a hydrogen atom or an SO₃H residue. The compounds of general formula I as well as compounds II and III can also be in form of their physiologically acceptable alkaline metal, alkaline earth metal and other salts, e. g. potassium salts. Also these salts are subject of the present invention.

Furthermore, plant extracts, in particular from Pelargonium species containing one or more compounds of general formula I, wherein at least one of the residues R⁶, R⁷ and R⁸ is an SO₃H residue, and the pharmaceutical preparations produced thereform form part of the present invention. Thereby, those extracts having a concentration of at least one of the compounds of general formula I in the dry matter proportion of the plant extract between 0.1% and 10% are preferred with those having a concentration between 0.5% and 5% being particularly preferred. The dry matter proportion corresponds to the dry residue according to Ph. Eur. (fluid extracts), wherein the analysis can also be effected directly, for example in the fluid extract and the dry residue can be considered by calculation.

The preparation of compound II can be effected by hydrolysis and/or ether cleavage, for example of commercially available fraxin or of a compound of general formula I, wherein at least one of the residues R⁶, R⁷ and R⁸ is an SO₃H residue.

The preparation of those compounds of general formula I wherein at least one of the residues R⁶, R⁷ and R⁸ is an SO₃H residue can be effected by reacting compound II with sulfur trioxide-trimethylamine complex or, in case of compound III, by isolation from suitable plant material, for example from dried roots of Pelargonium sidoides. The compounds 6,7-dihydroxy-8-sulfooxy-2H-1-benzopyran-2-one (general formula I; R⁶═R⁷═H; R⁸═SO₃H) and 7,8-dihydroxy-6-sulfooxy-2H-1-benzopyran-2-one (general formula I; R⁷═R⁸═H; R⁶═SO₃H) can also be obtained by partially hydrolysing compound III.

The extracts according to the present invention can be obtained in variable compositions from pelargonium plants or parts thereof by known preparation methods using solvents such as water, methanol, ethanol, acetone etc. and mixtures thereof at temperatures from room temperatures to 60° C. under slight to vigorous mixing or by percolation within 10 min. to 24 h. Preferred extractions solvents are water or mixtures of ethanol and water with a water proportion of at least 50% by weight, particularly preferred in a ratio of ethanol/water from 10/90 to 15/85 (w/w). In order to further concentrate the compounds of general formula I according to the present invention additional concentrations can be carried out, such as liquid-liquid distribution using for example 1-butanol/water or ethyl acetate/water, adsorption-desorption using ion exchangers, LH20, HP20 and other resins or chromatographic separations using RP18, silica gel and the like. If desired, further processing to obtain dry extracts is carried out according to methods known per se by removing the solvent at increased temperature and/or reduced pressure or by freeze-drying. According to the European Pharmacopoeia dry extracts generally have a dry residue of at least 95% by weight.

The compounds of general formula I according to the present invention and the extracts containing at least one of these compounds, respectively, can be administered preferably orally in form of powders, granules, tablets, dragees or capsules or as a solution.

The dosage is effected such that 0.1 mg per day to 250 mg per day, preferably 0.3 mg per day to 50 mg per day of one or more of the compounds of general formula I is administered.

For the preparation of tablets at least one of the compounds of general formula I or the corresponding extract is mixed with suitable pharmaceutically acceptable adjuvants such as lactose, cellulose, silicon dioxide, croscarmellose and magnesium stearate and pressed into tablets which are optionally provided with a suitable coating made of, for example, hydroxylmethylpropylcellulose, polyethylene glycol, colorants (e. g. titanium oxide, iron oxide) and talcum.

The efficacy of compound II in case of pathological conditions associated with oxidative stress and/or inflammatory reactions are supported by the experiments described in the following.

Antioxidative Properties:

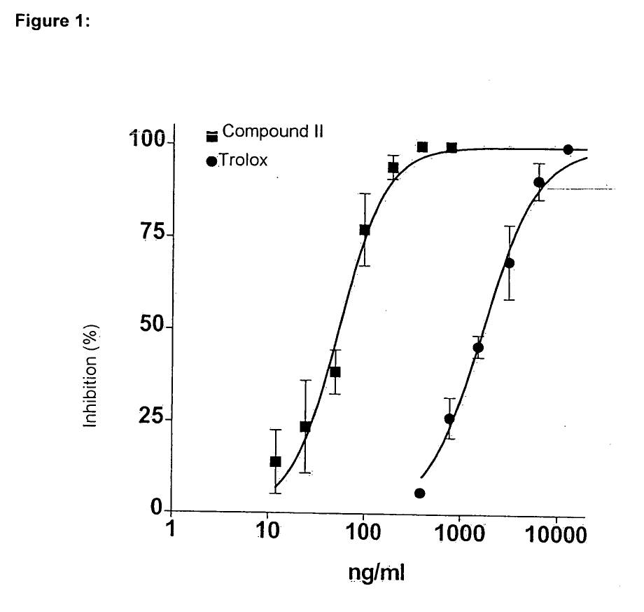

The autoxidation of lipids is associated with the emission of light. The determination of this extraordinarily weak chemiluminescence can be used for both quantifying peroxides and evaluating the efficacy of antioxidants. Brain tissue of male mice (NMRI; 20-30 g; Centre d'Elevage Janvier, Le Genest-Saint Isle, France) served as lipid-rich tissue in the present investigations. After its extraction the brain was washed with ice-cold phosphate-buffered physiological saline solution (PBS, pH 7.4) and freed from meninges and residual blood. The tissue samples were homogenised with 4 times their volume (v/w) made up of PBS and centrifugated at 1000×g and 4° C. for 10 minutes. The supernatants were immediately diluted with the same buffer to 3 times their volume and stored on ice. 250 μl of the diluted supernatant was transferred into a test tube and incubated for 10 minutes at 37° C. in a 6-channel luminometer (Multi-Biolumat LB 9505 C, Berthold, Bad Wildbad). After adding 25 μl of compound II in PBS added with 2.5% DMSO the incubation was continued for further 10 minutes. Then the intensity of the chemiluminescence (CL) was determined over a period of 60 minutes. The percentage of the inhibition of the autoxidation was calculated in comparison to the solvent control (PBS added with 2.5% DMSO) measured simultaneously. Compound II inhibited the autoxidation of the lipids with superior potency at a half-maximal inhibitory concentration of 53 ng/ml (FIG. 1). In contrast, Trolox, which is frequently used as a reference substance in determinations of antioxidative properties, only showed a half-maximal inhibitory concentration of 1665 ng/ml.

FIG. 1 shows the influence of compound II and Trolox on the autoxidation of lipids. The percentage of inhibition of the lipid peroxidation compared to a solvent control from three independent tests (average value ±SD) is stated.

Inhibition of the Synthesis of Proinflammatory Cytokines:

The influence of compound II on the synthesis of the proinflammatory cytokines IL-1β, TNF-α and IL-6 was determined by using activated murine peritoneal macrophages. In order to recover the activated macrophages 3×10⁹ killed coryn bacterium parvum bacteria (Changzhou Yanshen Co. Ltd., Changzhou, China) in 0.5 ml PBS were injected intraperitoneally into male NMRI mice (Centre d'Elevage Janvier, Le Genest-Saint Isle, France). 6 days later the abdominal cavity was rinsed with 2.5 ml Hanks' balanced saline solution (HBSS) free of calcium and magnesium added with 10 U/ml heparine. The cells were resuspended at a concentration of 2×10⁶ cells/ml in complete RPMI medium supplemented with 10% fetal bovine serum. 200 μm cell suspension were filled into the wells of 96-well microtiter plates, respectively. After an incubation period of 2 h non-adherent cells were removed and the remaining cell lawn was washed twice with culture medium (37° C.). The macrophages were preincubated for 30 min. with compound II and then the synthesis of proinflammatory cytokines was induced by adding 1 μg/ml lipopolysaccharide of E. coli (serotype 0127:B8, Sigma, Deisenhofen). After incubating for 24 h (37° C., 5% CO₂ in air) the cells were lysed by freezing and thawing for three times, the cell supernatants were recovered and frozen at −80° C. until analysed. The determination of the cytokine concentration in the cell supernatant was effected by means of commercial test kits (Duosets IL-1β, TNF-α and IL-6, R&D, Wiesbaden) in correspondence with the manufacturer's instructions. All investigations were conducted three times. The influence of compound II on the synthesis of the cytokines was evaluated in comparison to solvent controls (0.1% DMSO in complete RPMI medium) that were tested simultaneously. As can be seen from Table 1 below, compound II at a concentration of 100 μg/ml effected a significant inhibition of the synthesis of all three measured cytokines with the effect on the production of IL-6 being marked the strongest.

TABLE 1
The influence of compound II on the synthesis of proinflammatory
cytokines in activated mouse-peritoneal macropages. The average
values ± SD from three parallel tests are shown. The effect of
compound II was calculated as the percentage change in comparison
to a solvent control (*error probability P < 0.05, t-test).

| | Test | | | | | |
|---|---|---|---|---|---|---|
| | IL-1β | | TNF-α | | IL-6 | |
| | | Effect | | Effect | | Effect |
| | pg/ml | (%) | pg/ml | (%) | pg/ml | (%) |
| Control | 3553 ± | | 8393 ± | | 31449 ± | |
| | 293 | | 923 | | 2201 | |
| Compound II | 430 ± | −88* | 6251 ± | −26* | 976 ± | −97* |
| 100 μg/ml | 65 | | 7 | | 244 | |
| Compound II | 3356 ± | −6 | 10189 ± | +21 | 22519 ± | −28* |
| 30 μg/ml | 87 | | 1018 | | 3153 | |
| Compound II | 3789 ± | +7 | 10050 ± | +19 | 23078 ± | −27* |
| 10 μg/ml | 72 | | 462 | | 3461 | |

Inhibition of Cyclooxygenase and Lipoxygenase Activity in Human Whole Blood:

Heparinised human whole blood was used for the investigations. 100 μl of whole blood were added into each well of 96-well microtiter plates. Separated plates were used for the determination of the cyclooxygenase-1 (COX1) and lipoxygenase (LO) activiy as well as for the induction of cyclooxygenase-2 (COX2).

Compound II was diluted in DME medium (DMEM) with 1% of antibiotics/antimycotics solution and 2 mM L-glutamine (Sigma, Deisenhofen) using DMSO (final concentration 0.1%) as solubility promoter. After adding 50 μl of compound II the tests were incubated for 60 min. at 37° C. Subsequently, 50 μl of calciumionophor A23187 (final concentration 50 μM) were added to stimulate the eicosanoid synthesis. After incubating for further 30 min. at 37° C. the microtiter plates were centrifuged for 5 min. at 4° C. with 1500 g. The plasma was pipetted off and frozen at −80° C. until analysed.

In order to prove the COX2 activity the blood samples (100 μl/well) were initially pretreated with aspirin (50 μl in DMEM, final concentration 12 μg/ml) for 6 h at 37° C. to inactivate COX1. Then compound II and the solvent (DMEM with 0.1% DMSO), respectively, were added in a volume of 25 μl. Moreover, to induce the expression of COX2 25 μl of lipopolysaccharide of E. coli (serotype 0127:B8, final concentration 10 μg/ml) were added. After incubating for 18 h at 37° C. the plasma was recovered as described above and also stored at −80° C. until analysed.

In the plasma samples TXB₂, PGE₂ and cystenyl leukotrienes (cystenyl-LT) were determined as parameters for the COX1, COX2 and LO activity. For the analysis commercial EIA test kits (TXB2 and PGE2: Caymann/IBL, Hamburg; cystenyl leukotrienes: CAST-2000, Milenia, Bad Nauheim) were used in accordance with the manufacturers' instructions. It becomes clear from the results (cf. Table 2) that compound II effects a potent inhibition of the activity of COX2 and LO. The activity of COX1, on the other hand, is hardly affected. This spectrum of efficacy is to be regarded as extremely advantageous because in the therapeutic use of compound II the side effects typical of COX1 inhibitors, such as gastrointestinal complications (erosions, ulcerations) or hemorrhages due to the inhibition of the thrombocyte aggregation need therefore not to be taken into consideration.

TABLE 2
The influence of compound II on the synthesis of cystenyl-LT, TXB₂
and PGE₂ in human whole blood. The average values ± SD from two
parallel tests are shown. The effect of compound II was calculated
as a percentage change in comparison to a solvent control.

| | Test | | | | | |
|---|---|---|---|---|---|---|
| | Cystenyl-LT | | TXB₂ | | PGE₂ | |
| | | Effect | | Effect | | Effect |
| | pg/ml | (%) | pg/ml | (%) | pg/ml | (%) |
| Control | 7227 ± | | 9777 ± | | 10773 ± | |
| | 612 | | 1389 | | 944 | |
| Compound II | 661 ± | −91 | 9115 ± | −7 | 2232 ± | −79 |
| 100 μg/ml | 520 | | 244 | | 68 | |
| Compound II | 2089 ± | −71 | 9993 ± | +2 | 2626 ± | −76 |
| 30 μg/ml | 90 | | 6658 | | 503 | |
| Compound II | 3557 ± | −51 | 7825 ± | −20 | 3679 ± | −66 |
| 10 μg/ml | 86 | | 881 | | 41 | |
| Compound II | 5193 ± | −28 | 7278 ± | −26 | 5499 ± | −49 |
| 10 μg/ml | 542 | | 430 | | 86 | |

### EXAMPLE 1

### Preparation of 6,7,8-Trihydroxy-2H-1-benzopyran-2-one (Compound II)

20 g (42.7 mmol) 6,8-bis(sulfooxy)-7-hydroxy-2H-1-benzopyran-2-one potassium salt were stirred in 480 ml of about 2 N hydrochloric acid for 20 h at 40 to 50° C. After cooling the precipitated crude product was filtered off and recrystallised from water (hot filtration). The crystallizate was filtered off, washed and dried in vacuum at 100° C.: 5.9 g (71%), melting point: decomposition starting at 260° C.; ¹H and ¹³CNMR comply with the indications of O. Kayser and H. Kolodziej (Phytochemistry 39, 1181-1185 (1995)).

### EXAMPLE 2

### Isolation and Structure Determination of 6,8-Bis(sulfooxy)-7-hydroxy-2H-1-benzopyran-2-one Potassium Salt (Potassium Salt of Compound III)

15 kg of ground roots of Pelargonium sidoides were percolated twice at room temperature with 75 l and 40 l of water, respectively. The aqueous extract was concentrated to approximately 1/3, 7 kg of ammonium sulphate were added thereto and it was extracted several times with a 3/2 mixture of 2-butanone/ethanol. The organic phases were combined and concentrated by evaporation.

This residue was chromatographed over an HP20 column (eluent: water). The 6,8-bis(sulfooxy)-7-hydroxy-2H-1-benzopyran-2-one fractions were concentrated, adjusted to pH 8 using potassium hydroxide solution and diluted with ethanol in a ratio of 1/1. The precipitate was filtered off and suspended in water. Adjustment to pH 10.7 was conducted with potassium hydroxide solution and dilution was effected with ethanol in a ratio of 1/1. The precipitate settled as a result thereof was redissolved in hot water. The hot solution was filtered and diluted with ethanol in a ratio of 1/1. The settling crystallizate was filtered off, washed and dried in vacuum at 50° C.: 27.6 g (0.14% with respect to the plant material, calculated as the free acid).

Melting point: Decomposition starting at 216° C.; C₉H₃K₃O₁₁S₂ (468.55) found: C 23.08%, H 0.70%, K 24.65%, S 13.9%—calculated: C 23.07%, H 0.65%, K 25.04%, 037.56%, S 13.69%; ¹HNMR (DMSO-d₆): δ=7.62 (d, J=9.1 Hz, H-4), 7.03 (s, H-5), 5.59 (d, J=9.1 Hz, H-3); ¹³CNMR (DMSO-d₆): δ=162.9 (C-7), 161.9 (C-2), 147.7 (C-8), 145.1 (C-4), 142.2 (C-6), 130.4 (C-8a), 115.5 (C-5), 102.4 (C-3), 101.5 (C-4a).

The acidic hydrolysis of trihydroxy coumarine disulfate potassium salt results in 6,7,8-trihydroxy coumarine (cf. Example 1). For a further determination of the structure compound III was derivatised according to the following reaction sequence:

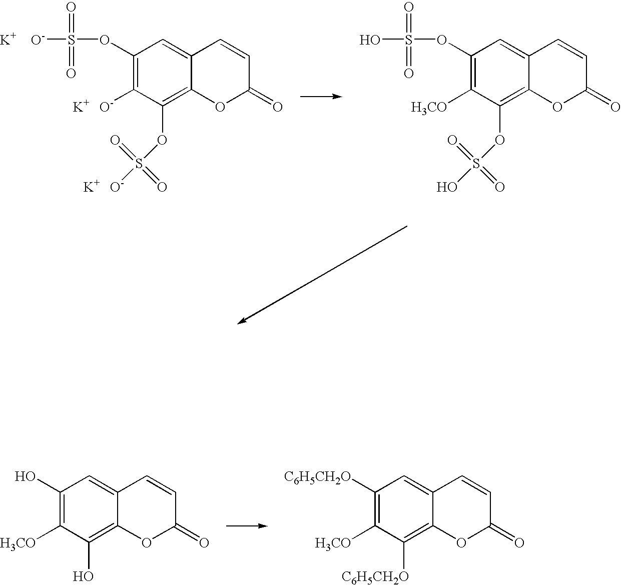

For this purpose the trihydroxy coumarine disulfate potassium salt was reacted with methyl iodide in the presence of potassium carbonate at 60° C. in DMF to yield the corresponding 7-methylether. After acidifying with concentrated hydrochloric acid the reaction mixture was stirred for 24 h at 50° C., extracted with ethyl acetate and chromatographed over silica gel (eluent: heptane/ethyl acetate 7/3): 6,8-dihydroxy-7-methoxycoumarine. This was reacted with benzyl bromide in the presence of potassium carbonate and potassium iodide in DMF at room temperature. The mixture was concentrated and the residue distributed between water and TBME. The organic phase was concentrated and chromatographed over silica gel (eluent: toluene/ethanol 95/5): 6,8-dibenzyloxy-7-methoxycoumarine.

The substitution pattern of the latter compound was determined with one-dimensional and two-dimensional NMR spectroscopy in CDCl₃. A clear NOESY correlation between H-5 and one of the. CH₂ signals allows an unambiguous conclusion of a benzyloxy residue in 6 position. Furthermore, the OCH₃ signal correlates with both CH₂ signals indicating that the methoxy group is positioned between the two benzyloxy residues, i.e. in 7-position. The HMBC correlations between C-7 and H-5 as well as OCH₃, as well as between C-8 and H-4 as well as 8-CH₂ confirm the substitution pattern taken from the NOESY. It can be clearly derived from the preparation sequence of the investigated derivative and its structure that the sulfoxy residues of the trihydroxy coumarine disulfate are bound in the positions 6 and 8.

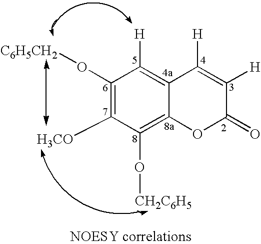

### EXAMPLE 3

### Plant Extract with a Content of 6,8-Bis(sulfooxy)-7-hydroxy-2H-1-benzopyran-2-one (Compound III)

500 g of ground roots of Pelargonium sidoides were extracted with 3 kg of water for 4 h at room temperature. The extracted plant material was filtered off and extracted once more with 2 kg of water as above and filtered. The filtrates were combined, concentrated at about 35° C. and freeze-dried: 58.7 g (11.7%) dry extract with a content of compound III of 1.54%.

### EXAMPLE 4

### Plant Extract with a Content of 6,8-Bis(sulfooxy)-7-hydroxy-2H-1-benzopyran-2-one (Compound III)

About 1.25 kg of ground roots of Pelargonium sidoides were extracted with about 12.5 kg of ethanol/water 11/89 (w/w) at room temperature. After filtration the filtrate was concentrated at about 45° C. and freeze-dried: 90.4 g (7.2%) dry extract with a content of compound III of 1.86%.

### EXAMPLE 5

### Tablets

For the preparation of tablets containing 5 to 250 mg of the active ingredient depending on the desired efficacy the following is required:

| | | |
|---|---|---|
| Compound II | 200 to 5 000 | g |
| Cellulose powder | 2 000 | g |
| Corn starch | 1 200 | g |
| Colloid silicic acid | 80 | g |
| Magnesium stearate | 20 | g |
| Lactose | to 10 000 | g |

The active ingredient is optionally ground, homogeneously mixed with the adjuvants and pressed into tablets having a weight of 250 mg and a diameter of 9 mm, respectively, in the conventional way. At dosages exceeding 125 mg tablets having a weight of 500 mg and a diameter of 11 mm, respectively, are pressed. If desired, the tablets are provided with a film coating.

1-22. (canceled)

23. A method for treating a subject suffering from or susceptible to a pathological condition associated with oxidative stress and/or inflammatory reaction, comprising administering to the subject one or more compounds of the following formula I: 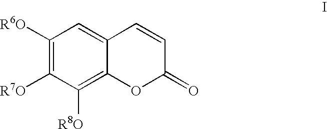
wherein R⁶, R⁷ and R⁸ independently represent H or SO₃H, and physiologically acceptable salts thereof.

24. A method of claim 23 wherein R⁶, R⁷ and R⁸ are hydrogen atoms.

25. A method of claim 23 wherein R⁶ and R⁸ represent SO₃H and R⁷ represents hydrogen.

26. A method of claim 23 wherein one or more of the compounds of formula I are contained in a plant extract.

27. A method of claim 23 wherein a plant extract comprising one or more of the compounds of formula I is administered to the subject.

28. A method of claim 26 wherein the plant extract is an extract from Pelargonium species.

29. A method of claim 28 wherein the plant extract is an extract from Pelargonium sidoides.

30. A method of claim 26 wherein the concentration of at least one of the compounds of formula I in the dry content of the plant extract is between 0.1% and 10%.

31. A method of claim 26 wherein the concentration of at least one of the compound(s) of formula I in the dry content of the plant extract is between 0.5% and 5%.

32. A method of claim 23 wherein the patient is suffering from or susceptible to diabetes mellitus type I and/or II; rheumatoid arthritis; asthma; colitis ulcerosa; Morbus Crohn; psoriasis; neurodermitis; bacterial infection; virus comprising influenza; AIDS; viral hepatitis; pathogen comprising parasite; pathogen comprising fungi; pathogen comprising prions; artherosclerosis; endothelial dysfunction; ischemia; neurologic disease; neurodegenerative disease; cataract; and/or tumor disease.

33. A method of claim 23 wherein the patient is suffering from or susceptible to diabetes mellitus type I and/or II; rheumatoid arthritis; asthma; colitis ulcerosa; Morbus Crohn; psoriasis; neurodermitis; bacterial infection; virus comprising influenza; AIDS; viral hepatitis; pathogen comprising parasite; pathogen comprising fungi; pathogen comprising prions; artherosclerosis; endothelial dysfunction; ischemia; Morbus Alzheimer; Morbus Parkinson; neurodegenerative disease; cataract; and/or tumor disease.

34. A method of claim 23 wherein the patient is suffering from or susceptible to diabetes mellitus type I and/or 11; rheumatoid arthritis; asthma; colitis ulcerosa; Morbus Crohn; psoriasis; neurodermitis; bacterial infection; virus comprising influenza; AIDS; viral hepatitis; pathogen comprising parasite; pathogen comprising fungi; pathogen comprising prions; artherosclerosis; endothelial dysfunction; ischemia; Morbus Alzheimer; Morbus Parkinson; neurodegenerative disease; cataract; and/or tumor disease.

35. A method of claim 23 wherein the patient is suffering from diabetes mellitus type I and/or II; rheumatoid arthritis; asthma; colitis ulcerosa; Morbus Crohn; psoriasis; neurodermitis; bacterial infection; virus comprising influenza; AIDS; viral hepatitis; pathogen comprising parasite; pathogen comprising fungi; pathogen comprising prions; artherosclerosis; endothelial dysfunction; ischemia; Morbus Alzheimer; Morbus Parkinson; neurodegenerative disease; cataract; and/or tumor disease.

36. A compound of the following formula I: 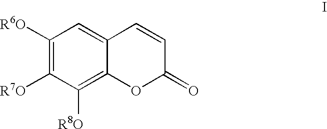
wherein R⁶, R⁷ and R⁸ independently represent H or SO₃H, and physiologically acceptable salts thereof,
provided that R⁶, R⁷ and R⁸ are not H at the same time.

37. A compound of claim 36 wherein
R⁶ and R⁸ represent SO₃H and R⁷ represents hydrogen, or
R⁶ represents SO₃H and R⁷ and R⁸ represent hydrogen, or
R⁸ represents SO₃H and R⁶ and R⁷ represent hydrogen.

38. A plant extract comprising one or more of the compounds of claim 36.

39. A plant extract of claim 38 wherein the plant extract is an extract from Pelargonium species.

40. A plant extract of claim 38 wherein the plant extract is an extract from Pelargonium sidoides.

41. A plant extract of claim 38 wherein the concentration of at least one of the compounds in the dry content of the plant extract is between 0.1% and 10%.

42. A plant extract of claim 41 wherein the concentration of at least one of the compounds in the dry content of the plant extract is between 0.5% and 5%.

43. A medicament for the treatment or prophylaxis of pathological conditions associated with oxidative stress and/or inflammatory reactions comprising one or more compounds of formula I of claim 23.

44. The medicament of claim 43 wherein R⁶, R⁷ and R⁸ are not all H.

45. A dietetic food product for supporting the treatment or for the prophylaxis of pathological conditions associated with oxidative stress and/or inflammatory reactions comprising one or more compounds of formula I of claim 23.

46. A medicament of claim 43 wherein R⁶, R⁷ and R⁸ are not all H.

47. A pharmaceutical preparation comprising one or more compounds of formula I of claim 23 and one or more adjuvants as an oral administration form.

48. A pharmaceutical preparation of claim 47 wherein R⁶, R⁷ and R⁸ are not all H.

49. A pharmaceutical preparation comprising a plant extract of claim 38 and one or more adjuvants as an oral administration form.
